(19) United States
(12) Patent Application Publication
(10) Pub. No.: US 2003/0171642 A1
(43) Pub. Date: Sep. 11, 2003

(54) Intra-aortic balloon catheter having a releasable guide wire

(76) Inventors: Robert B. Schock, Sparta, NJ (US); Daniel A. Walters, Township, NJ (US)

Correspondence Address: Datascope Corp., 14 Philips Parkway, Montvale, NJ, 07645, US

(21) Appl. No.: 10/091,073

(22) Filed: Mar. 5, 2002

(51) Int. Cl. A61M 31/00

(52) U.S. Cl. 600/18; 604/103.04

(57) ABSTRACT

A single lumen monorail intra-aortic balloon catheter and guide wire system. The catheter and guide wire being advanced through a patient's vasculature as a unit. The guide wire comprising a tip guide section and a pull section configured so as to minimize bleeding at the insertion site.

### BACKGROUND OF THE INVENTION

1. Field of the Invention

The invention relates to a balloon catheter. More particularly, the invention relates to a single lumen monorail intra-aortic balloon (IAB) catheter having a specialized guide wire for minimizing blood leakage.

2. Description of the Prior Art

IAB catheters are used in patients with left heart failure to augment the pumping action of the heart. The catheters, approximately one (1) meter long, have an inflatable and deflatable balloon at the distal end. The catheter is typically inserted into the femoral artery and moved up the descending thoracic aorta until the distal tip of the balloon is positioned just below or distal to the left subclavian artery. The proximal end of the catheter remains outside of the patient's body. A passageway for inflating and deflating the balloon extends through the catheter and is connected at its proximal end to an external pump. The patient's central aortic pressure is used to time the balloon and the patient's ECG may be used to trigger balloon inflation in synchronous counterpulsation to the patient's heart beat.

IAB therapy increases coronary artery perfusion, decreases the workload of the left ventricle, and allows healing of the injured myocardium. Ideally, the balloon should be inflating immediately after the aortic valve closes and deflating just prior to the onset of systole. When properly coordinated, the inflation of the balloon raises the patient's diastolic pressure, increasing the oxygen supply to the myocardium; and balloon deflation just prior to the onset of systole lowers the patient's diastolic pressure, reducing myocardial oxygen demand.

IAB catheters may also have a secondary passageway or lumen which can be used to measure aortic pressure. In this dual lumen or co-lumen construction, the secondary lumen may also be used to accommodate a guide wire to facilitate placement of the catheter and to infuse fluids, or to do blood sampling.

Typical dual lumen IAB catheters have an outer, flexible, plastic tube, which serves as the inflating and deflating gas passageway, and a central tube therethrough formed of plastic tubing, stainless steel tubing, or wire coil embedded in plastic tubing. A polyurethane compound is used to form the balloon. Other IAB catheters on the market have a co-lumen configuration wherein the inner tube is connected to or embedded in the outer tube wall.

A great deal of effort has been exerted in an effort to reduce the outer diameter of the dual or co-lumen IAB catheter. A reduction in size is desired in order to minimize the size of the arterial opening, to facilitate percutaneous insertion of the catheter into the aorta, maximizing blood flow past the inserted catheter, and also to allow for the use of a smaller insertion sheath to further maximize distal flow. Progress has certainly been made: IAB catheters currently on the market have outer diameters of as low as 8.0 Fr compared to over 10.0 Fr only a few years ago. Progress has been incremental, however, because of the difficulties encountered in reducing component sizes while still maintaining the necessary physical design requirements of the overall catheter required for efficient counterpulsation therapy and for smooth percutaneous insertion into the patient's blood vessel.

In order to further reduce size, the present invention involves a complete shift in the design of the IAB from a dual lumen device to a single lumen monorail device. In the past, a single lumen design for an IAB catheter may not have been workable given the necessity of the secondary lumen for blood pressure monitoring. Recently, however, Datascope Corp. has made advancements in tip sensor technology allowing for elimination of the inner lumen. See U.S. patent application Ser. Nos. 09/735,076, 09/734,755, and 09/925,143, filed on Dec. 11, 2000, Dec. 11, 2000, and Aug. 9, 2001, respectively, all herein incorporated by reference in their entirety.

Reduction of the cross sectional profile of the wrapped balloon has also been a major design obstacle of single lumen IAB catheter design. Elimination of the inner lumen allows for a reduction in the size of the catheter, however, it does not assure that the cross sectional profile of the wrapped balloon membrane has an equal or lower cross section profile than the catheter. Recently, however, Datascope Corp. has made advancements in reduction of thickness of the balloon membrane. See U.S. Pat. No. 6,213,975 and U.S. patent application Ser. No. 09/757,859, filed on Jan. 10, 2001, both herein incorporated by reference in their entirety. Accordingly, solutions to a number of major IAB catheter design impediments now allow for the potential design of a single lumen IAB.

Use of a single lumen monorail type IAB is known for pediatric purposes, see for example U.S. Pat. No. 6,146,372, herein incorporated by reference in its entirety. Despite the problems outlined above, specifically the lack of a central lumen to monitor blood pressure, a single lumen monorail type IAB was and is the only catheter small enough to fit within a pediatric patient's blood vessel. In order to make up for this deficiency, blood pressure in pediatric patients undergoing IAB therapy is taken from an alternate source such as a catheter in the radial artery. The balloon membrane thickness does not pose as much of a design problem as it does for adult IAB catheters because the inflated profile is disproportionately smaller for the inflated pediatric balloon allowing for a wrapped balloon membrane cross sectional profile equivalent to the remainder of the catheter. Given the drawbacks with the pediatric single lumen monorail IAD catheter a monorail design has not yet been implemented for adults having blood vessels large enough to accommodate a dual lumen catheter.

Another major problem with IAB single lumen monorail catheters involves excessive bleeding at the insertion site. The monorail catheter is typically advanced over a preinserted guide wire into the blood vessel. Upon placement the exposed proximal ends of both the catheter and guide wire at the insertion site contact each other. The catheter and guide wire, given their round cross sectional shape, and the tissue tract create a passageway through which blood escapes.

While the present state of the art IAB catheters may be suitable for the particular purpose employed, or for general use, they would not be as suitable for the purposes of the present invention as disclosed hereafter.

### SUMMARY OF THE INVENTION

The invention is single lumen monorail IAB catheter and guide wire system and method for inserting same. The catheter and guide wire are advanced as a single unit through the vasculature of the patient.

The guide wire comprises a distal tip guide section and a proximal pull section. The pull section allowing for the tip guide section to be pulled out after final positioning of the catheter and having an optimal cross sectional area and size, is generally smaller than that of the tip guide section so as to assure minimum blood leakage at the insertion site. The tip guide section is capable of guiding the catheter through a patients vasculature.

To the accomplishment of the above and related objects the invention may be embodied in the form illustrated in the accompanying drawings. Attention is called to the fact, however, that the drawings are illustrative only. Variations are contemplated as being part of the invention, limited only by the scope of the claims.

### BRIEF DESCRIPTION OF THE DRAWINGS

In the drawings, like elements are depicted by like reference numerals. The drawings are briefly described as follows.

FIG. 1 is longitudinal cross sectional view of the single lumen monorail IAB catheter of the present invention inserted through a tissue tract into a blood vessel of a patient.

FIG. 1A is a transverse cross sectional view of IAB catheter and guide wire of FIG. 1 at the insertion site taken along lines 1A-1A.

FIG. 1B is a transverse cross sectional view of an IAB and prior art guide wire taken at the insertion site.

FIG. 1C is the transverse cross sectional view of FIG. 1A with an alternate flat pull section.

FIG. 2A is a longitudinal cross sectional view of an angiographic needle inserted through a tissue tract into a blood vessel of a patient.

FIG. 2B is a longitudinal cross sectional view of the needle of FIG. 2A with a guide wire inserted through it into the blood vessel.

FIG. 2C is a longitudinal cross section view of an insertion sheath and dilator assembly inserted into blood vessel 12 and the catheter 10 just prior to complete insertion into blood vessel 12.

FIG. 3 illustrates a side view of catheter 10, into which guide wire 24 has been pre-loaded in preparation for insertion.

FIG. 4A is a longitudinal cross section of a prior art guide wire.

FIG. 4B is a longitudinal cross section of the preferred embodiment of the guide wire of the present invention.

FIG. 4C is a longitudinal cross section of a first alternate embodiment of the guide wire of the present invention.

FIG. 4D is a longitudinal cross section of a second alternate embodiment of the guide wire of the present invention.

FIG. 4E is a longitudinal cross section of a third alternate embodiment of the guide wire of the present invention.

FIG. 5 is a longitudinal cross section of a first alternate guide wire tip.

FIG. 6 is a longitudinal cross section of a second alternate guide wire tip.

### DETAILED DESCRIPTION OF THE PREFERRED EMBODIMENTS

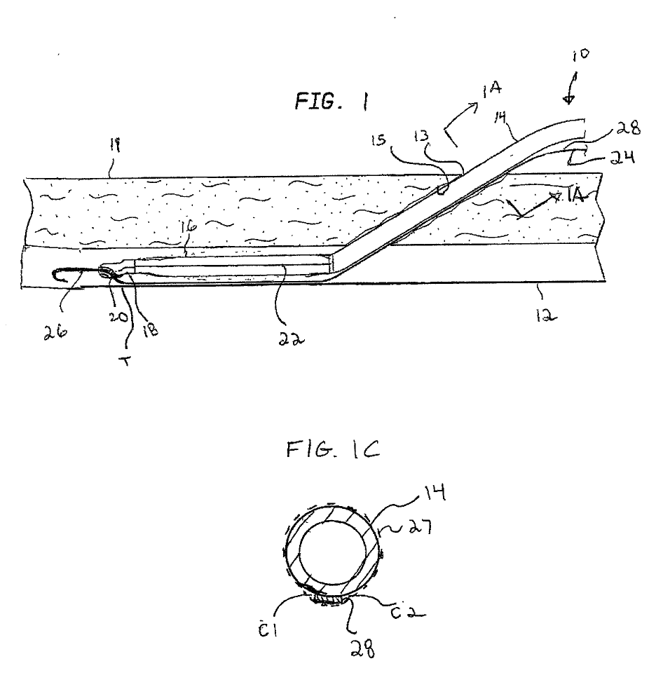

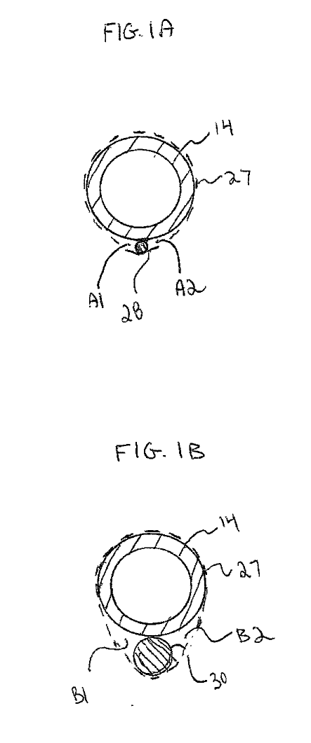

FIG. 1 illustrates the single lumen monorail IAB catheter of the present invention, generally designated 10, partially advanced into the blood vessel 12 of a patient 11 through insertion site 13 and tissue tract 15. IAB catheters are generally inserted through the femoral artery but can be inserted through other blood vessels as well, including the radial artery. Catheter 10 comprises a catheter tube 14 connected on a distal end to a proximal end of a balloon membrane 16. Said balloon membrane 16 being connected on a distal end to a tip 18 having a lumen 20. A stylet 22 extends from the distal end of catheter tube 14 to tip 18. Catheter tube 14 may optionally contain a removable stylet, as disclosed in U.S. patent application Ser. No. 09/813,905, filed on Mar. 21, 2001, herein incorporated by reference in its entirety, for enhancing the stiffness of catheter 10 during insertion. A guide wire 24 is removably disposed within lumen 20 of tip 18. Gas, preferably Helium, pumped through catheter 10 and into balloon membrane 16 is used to repeatedly inflate and deflate balloon membrane 16.

Tip 18 has a flattened [symbol] S [symbol] shape and is made from either silicone or polyurethane, both of which display a special property: If a needle is pulled through a piece of either of these materials any hole or lumen formed will self occlude. Details regarding tip 18 are disclosed in U.S. Pat. No. 6,146,372, herein incorporated by reference in its entirety. The self occluding property may help prevent blood from accumulating and possibly forming embolus in lumen 20.

Catheter tube 14 may have an outer diameter of between approximately 0.065 [symbol] (1.67 mm) and 0.131 [symbol] (3.33 mm), but preferably is approximately 0.084 [symbol] (2.13 mm). Catheter tube 14 may have a thickness of between approximately 0.004 [symbol] (0.10 mm) and 0.012 [symbol] (0.030 mm), but preferably has a thickness of approximately 0.006 [symbol] (0.15 mm). Balloon membrane 16 may have a single wall thickness of between 0.001 [symbol] (0.025 mm) and 0.006 [symbol] (0.15 mm), but preferably has a single wall thickness of 0.002 [symbol] (0.050 mm). Catheter tube 14 is preferably made from reinforced or unreinforced polyurethane, but may also be made with a variety of reinforced or nonreinforced biocompatible materials such as PTFE, FEP, PET, Pebax, nylon, Peek, polyimide, PVC, polyethylene, Nitinol™, silicone or any multi-layered combination of these materials. If catheter reinforcement is utilized, it is preferably accomplished with a stainless steel coil or braid embedded in a wall of the catheter tube 14. However, the reinforcement may alternatively be a coil or braid made of another suitably stiff, elastic material such as Nitinol, nylon, polyimide, or liquid crystal. Balloon membrane 16 is preferably made from polyurethane but may also be made from PET, silicone, PVC, polyethylene, or any other flexible biocompatible plastic.

Guide wires are traditionally used to assist guide catheters to a position in the vasculature necessary for therapy. They are designed so as to assure that they can be independently advanced into the patient's vasculature and guide a catheter, at least partially disposed about it, to a position in the patient's vasculature. Given that the guide wire is generally independently advanced into the vasculature it must be sufficiently stiff throughout its length to prevent buckling.

As indicated above, one major difficulty with a sheathless insertion of a catheter over a guide wire involves bleeding at the insertion site. The monorail catheter system of the present invention is inserted in a novel manner so as to allow for a guide wire having a proximal pull section 28 designed to minimize the bleeding at the insertion site. Relative to distal tip guide section 26 pull section 28 is much smaller in diameter, thus, minimizing the size of blood leakage channels created between tissue channel 15, catheter tube 14, and guide wire 24. In the novel insertion method, rather than being advanced over an already inserted and positioned guide wire, catheter 10 and guide wire 24 of the present invention are advanced together as a unit into the vasculature. Tip guide section 26 provides the necessary guidance to allow the catheter to track through the vasculature. Ideally, the portion of tip guide section 26 closest to catheter tip 18 is relatively stiff to support the catheter during insertion, while a most distal portion of tip guide section 26 is more flexible and incorporates a J-tip to facilitate passage through tortuous vessels. The catheter/guide wire combination has sufficient stiffness to be advanced into the vasculature.

The purpose of tip guide section 26 is to assist IAB catheter 10 to its preferred final position for pumping, just distal the left subclavian artery. The purpose of pull section 28 is to allow tip guide section 26 to be pulled out of lumen 20 and blood vessel 12 after final positioning of catheter 10 and prior to initiation of therapy.

The cross sectional geometry of pull section 28 is preferably configured to minimize the amount of blood loss at insertion site 13. FIG. 1A illustrates a transverse cross section of catheter tube 14 and pull section 28 wherein pull section 28 has a round cross sectional shape with a diameter of approximately 0.01 [symbol] (0.25 mm) and catheter tube 14 has a diameter of approximately 0.084 [symbol] (2.1 mm). Dotted line 27 disposed about catheter tube 14 and pull section 28 represents tissue tract 15 (FIG. 1). The blood leakage passageways created by tissue tract 15, pull section 28, and catheter tube 14 are designated A1 and A2. In contrast, FIG. 1B shows the cross sectional shapes of catheter tube 14 along side a traditionally sized guide wire 30 at the insertion site. Note that guide wire 30, approximately 0.020 [symbol] (0.50 mm), is considerably larger than pull section 28, approximately 0.010 [symbol] (0.25 mm), of the present invention. Accordingly, the blood passageway area created by catheter tube 14, guide wire 30, and tissue tract 15 (represented by dotted line 27), designated B1 and B2, is significantly larger than A1 and A2(FIG. 1A), thus allowing for more blood leakage through this area out of insertion site 13 (FIG. 1).

Note that pull section 28 does not have to be round nor have a specific size. See for example FIG. 1C, where pull section is a small section of a circle having a slightly larger diameter than catheter tube 14. C1 and C2 represent the blood leakage area. Pull section 28 may have any cross sectional shape and size so long as it minimizes the blood leakage area at insertion site 13. This can be accomplished, for example, by using a wire or string for pull section 28. The diameter of this wire or string will typically be smaller than the diameter of tip guide section 26 because tip guide section 26 must be sufficiently stiff to function properly as a support to the catheter tip 18 during insertion. Alternatively, pull section 28 may have another shape, including but not limited to, a rectangular, oval, V, U or semi or quarter circle cross section shape that tends to hug an outer surface of catheter tube 14. Given that tissue tract 15 generally will hug catheter tube 14 and pull section 28, the smaller pull section 28 is the smaller the blood leakage area will be.

FIG. 3 illustrates a side view of catheter 10, into which guide wire 24 has been pre-loaded in preparation for insertion. Tip guide section 26 is disposed within lumen 20 of tip 18 as shown in ghost lines. At point T the guide wire exits lumen 20 and transitions to pull section 28, which is preferably captured by the folds of wrapped membrane 16. Note that transition point T may be shifted proximally, lengthening tip guide section 26, however, the transition should always be inside patient 11, preferably inside blood vessel 12, when catheter 10 is finally positioned for therapy. At point P pull section 28 emerges from the folded membrane to lie along the outside of catheter tube 14. Pull section 28 then passes between catheter 14 and protective sleeve unit 33, exiting at point Y. Protective sleeve unit 33 maintains the cleanliness of catheter 10 and includes an expandable polymeric protective sleeve 34; the details of protective sleeve unit 33 are disclosed in U.S. patent application Ser. No. 09/347,868, filed on Jul. 6, 1999, herein incorporated by reference in its entirety. The pull section is optionally connected to torquing device 35, which is adjacent or attached to y-fitting 36. Torquing device 35 allows guide wire 24 to be rotated relative to catheter 10 during insertion, facilitating guidance of the entire device through the vasculature.

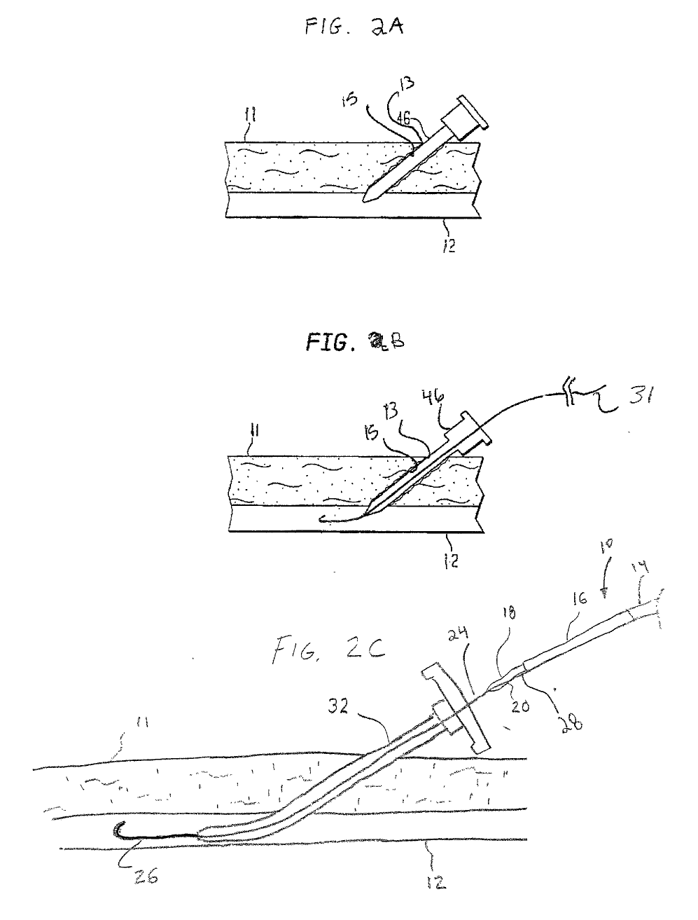

FIGS. 1, 2A, 2B, and 2C illustrate the insertion of catheter 10. FIG. 2A illustrates the end result of the first insertion step, namely insertion of an angiographic needle 46 through tissue tract 15 into blood vessel 12.

FIG. 2B illustrates preinsertion guide wire 31 inserted through needle 46 into blood vessel 12. Upon removal of needle 46, a splitable insertion sheath 32 and dilator (not shown) assembly is passed over first guide wire 31 into blood vessel 12. After insertion of this assembly the dilator and preinsertion guide wire 31 are removed. Tip guide section 26 of guide wire 24, which is pre-assembled to catheter 10, is next advanced into sheath 32, see FIG. 2C. Once the tip guide section 26 has entered the blood vessel 12, sheath 32 is removed from blood vessel 12, split, and discarded. Catheter 10 and guide wire 24 are then advanced into blood vessel 12 as a unit. Thus, despite use of the sheath dilator for dilation purposes, catheter 10 is inserted sheathlessly given that sheath dilator assembly 32 is removed prior to insertion of catheter 10 into blood vessel 12. The catheter/guide wire unit is positioned such that tip 18 is just distal the left subclavian artery. Guide wire 24 is then removed by pulling pull section 28 out of blood vessel 12 and therapy is initiated (balloon membrane 16 may need to be inflated to release guide wire 24 from wings of balloon membrane 16 folded over it). Note that if a stiffening stylet is disposed within catheter 14 during insertion, as disclosed in U.S. patent application Ser. No. 09/813,905, filed on Mar. 21, 2001, herein incorporated by reference, it must be removed prior to initiation of therapy.

Note that the present invention is not limited to intra-aortic balloon therapy. The principles of the present invention can be used with any catheter sheathlessly inserted with the aid of a guide wire.

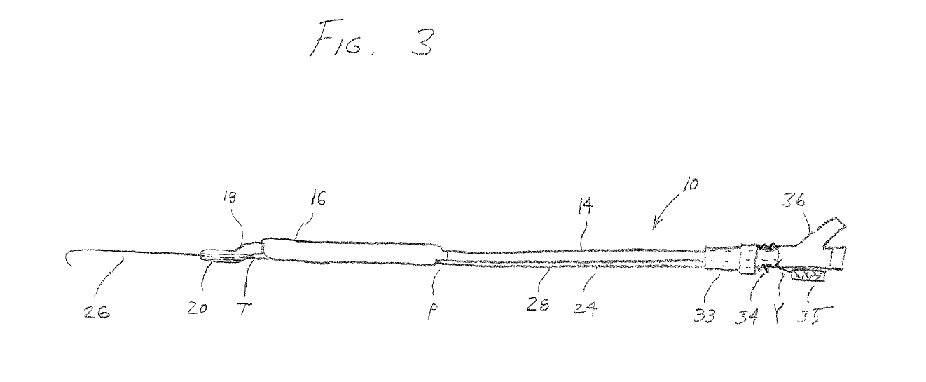

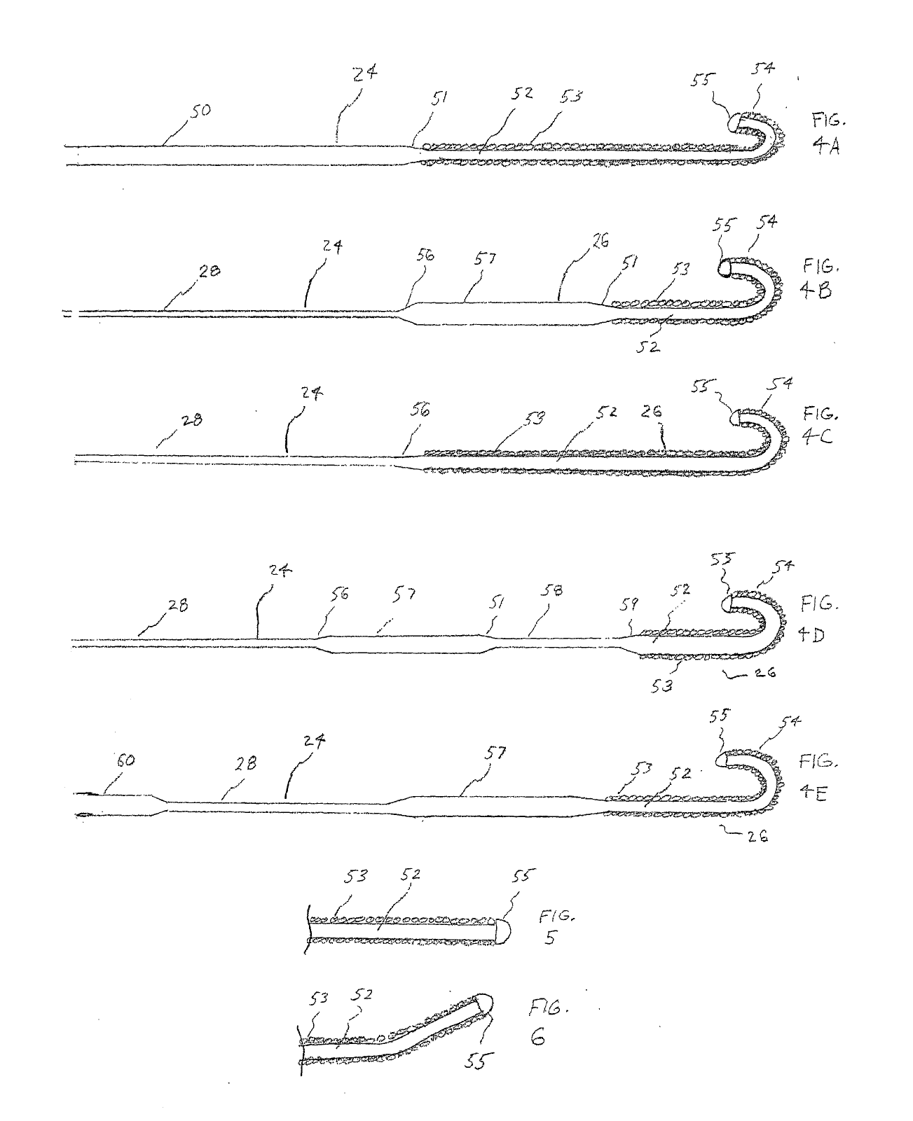

Guide wire 24 may have a variety of designs, but as discussed above, a novel approach is beneficial to this invention. FIG. 4A shows a guide wire 24 typical of the prior art as would be used with a conventional dual lumen IAB catheter. Prior art guide wire 24 incorporates a core wire with larger diameter proximal section 50 which transitions at taper 51 to a smaller-diameter distal core 52, which is bent into J-tip 54 and terminated with smooth bead 55. Overlying the distal core is coil 53. The larger diameter proximal core is required to allow the wire to be pushed unsupported through the vasculature.

FIG. 4B depicts the preferred embodiment of improved guide wire 24. The core wire incorporates a smaller diameter proximal pull section 28, which transitions into larger diameter distal section 57 at taper 56. Larger diameter distal section 57 transitions to smaller diameter distal section 52 at taper 51. The smaller diameter distal core section is bent into J-tip 54 and terminated with smooth bead 55. Optionally overlying the distal core is coil 53. Collectively, the guide wire features which are distal to taper 56 comprise distal tip guide section 26.

FIG. 4C shows a first alternate embodiment for improved guide wire 24. As illustrated, the core wire's small-diameter proximal pull section 28 transitions at taper 56 to a larger diameter distal core, the diameter of which is maintained through J-tip 54. This would be a less expensive design to produce than the design of FIG. 4B.

FIG. 4D shows a second alternate embodiment of improved guide wire 24. Its smaller diameter proximal pull section 28 transitions to larger diameter section 57, back down to smaller diameter section 58, and back up to larger diameter section 52. Additional transitions may be added, if desired. These transitions may be of use to optimize stiffness, and to allow for good fit of guide wire 24 to the IAB catheter 10.

FIG. 4E illustrates a third alternate embodiment of improved guide wire 24. This is similar to the design of FIG. 4B, except for the addition of larger diameter core section 60 at its most proximal end. This improves the ability of the portion of guide wire 24 which remains outside of the body to transmit torque to the portion of guide wire 24 which enters the body. The benefit of this structure is that it improves steerability of the guide wire 24.

FIG. 5 shows a first alternate tip design for improved guide wire 24. Rather than having a J-shape, this tip is straight. This design optimizes passage of guide wire 24 through very small vessels.

FIG. 6 shows a second alternate tip design for improved guide wire 24. This tip is a slightly bent [symbol] hockey stick [symbol] design, which enhances steerability of the wire through tortuous vessels.

Note that the alternate tip and wire constructions may be used in various combinations, as desired.

As many apparently widely different embodiments of the present invention can be made without departing from the spirit and scope thereof, it is to be understood that the invention is not limited to the specific embodiments thereof except as defined in the appended claims.

What is claimed is:

1. A single lumen intra-aortic balloon catheter system comprising a catheter and a guide wire for insertion as a unit through an insertion site and tissue track of a patient into a blood vessel of the patient,
said catheter comprising a catheter tube having distal and proximal ends, a balloon membrane having distal and proximal ends, and a tip having a lumen, the proximal end of the balloon membrane being connected to the distal end of the catheter tube and the distal end of the balloon membrane being connected to the tip,
said guide wire comprising a guide section and a more proximal pull section,
said pull section having a smaller cross sectional area than the guide section.

2. The catheter system as claimed in claim 1 wherein the guide section is stiffer than the pull section.

3. A single lumen intra-aortic balloon catheter system comprising a catheter and a guide wire for insertion as a unit through a tissue track into a blood vessel of a patient,
said catheter comprising a catheter tube having distal and proximal ends, a balloon membrane having distal and proximal ends, and a tip having a lumen, the proximal end of the balloon membrane being connected to the distal end of the catheter tube and the distal end of the balloon membrane being connected to the tip,
said guide wire comprising a guide section and a more proximal pull section,
when the catheter and pull section are placed side-by-side inside the tissue tract an outer surface of the catheter and pull section and the tissue tract define a first leakage area through which blood can flow, when the catheter and guide section are placed side-by-side inside the tissue tract an outer surface of the catheter and guide section and the tissue tract define a second leakage area through which blood can flow, the dimensions and cross sectional shape of the guide and pull sections being such that the first leakage area is smaller than the second leakage area.

4. The catheter system as claimed in claim 3 wherein the pull section has a smaller cross sectional area than the guide section.

5. The catheter system as claimed in claim 3 wherein the guide section is stiffer than the pull section.

6. A method for inserting a single lumen intra-aortic balloon catheter comprising a catheter tube having distal and proximal ends, a balloon membrane having distal and proximal ends, and a tip having a lumen, the proximal end of the balloon membrane being connected to the distal end of the catheter tube and the distal end of the balloon membrane being connected to the tip, a distal guide portion of a guide wire being disposed within the tip lumen and extending beyond a distal end of the tip, said method comprising the step of passing the catheter along with the guide wire as a unit through a tissue tract and into a blood vessel of a patient as a unit and advancing said unit to a position in the vasculature of the patient appropriate for pumping.

7. The method as claimed in claim 6 wherein the distal portion of the guide wire is passed through the tissue tract into the blood vessel via a preinserted insertion sheath.

8. The method as claimed in claim 6 comprising the additional step of pulling the guide wire out of the blood vessel while holding the catheter in place.

9. The method as claimed in claim 6 wherein the guide wire further comprises a pull section proximal to the guide section, said pull section having a smaller cross sectional area than the guide section.

10. The method as claimed in claim 6 wherein the guide wire further comprises a pull section proximal to the guide section, said guide section being stiffer than the pull section.

11. The method as claimed in claim 6 wherein the blood vessel is the femoral artery, axillary artery, brachial artery, or the radial artery.

12. A method for inserting a single lumen intra-aortic balloon catheter comprising a catheter tube having distal and proximal ends, a balloon membrane having distal and proximal ends, and a tip having a lumen, the proximal end of the balloon membrane being connected to the distal end of the catheter tube and the distal end of the balloon membrane being connected to the tip, a distal guide portion of a guide wire being disposed within the tip lumen and extending beyond a distal end of the tip, said method comprising the steps of: (a) passing a preinsertion guide wire into a patient's blood vessel; (b) passing an insertion sheath over said guide wire at least partially into the blood vessel; (c) removing the preinsertion guide wire; (d) inserting the distal portion of the guide wire into the blood vessel through the insertion sheath; (e) removing the insertion sheath from the blood vessel while leaving the distal portion of the guide wire within the blood vessel; (f) passing the catheter along with the guide wire as a unit into the blood vessel and advancing said unit to a position appropriate for pumping; and (e) pulling the guide wire out of the blood vessel while holding the catheter in place.

13. The method as claimed in claim 12 wherein the preinsertion guide wire is inserted into the blood vessel through an angiographic needle which is removed after insertion of the preinsertion guide wire.

14. The method as claimed in claim 12 wherein the guide wire further comprises a pull section proximal to the guide section, said pull section having a smaller cross sectional area than the guide section.

15. The method as claimed in claim 12 wherein the guide wire further comprises a pull section proximal to the guide section, the guide section being stiffer than said pull section.

16. The method as claimed in claim 12 wherein the insertion sheath comprises a sheath/dilator assembly.

17. The method as claimed in claim 12 wherein the blood vessel is the femoral artery or the radial artery.

18. A guide wire having a proximal section and a distal section, said distal section terminating in a tip, said proximal section having a smaller cross sectional area than the distal section.

19. The guide wire as claimed in claim 18 wherein the distal section is stiffer than the proximal section.

20. The guide wire as claimed claim 18 wherein the guide wire has a circular cross section having a diameter and wherein the diameter of the distal section is larger than the diameter of the proximal section.

21. The guide wire as claimed in claim 18 wherein a distal portion of the distal section has a j shaped hook shape.

22. The guide wire as claimed in claim 18 wherein a distal portion of the distal section has a hockey-stick shape.

23. A guide wire having a proximal section and a distal section, said distal section terminating in a tip, said proximal section having a higher stiffness than the distal section.

24. A guide wire having a proximal section, a first distal section, and a second distal section distal the first distal section and terminating in a tip, said first distal section having a larger cross sectional area than both the proximal section and the second distal section.

25. The guide wire as claimed in claim 24 wherein the second distal section has a larger cross sectional area than the proximal section.

26. The guide wire as claimed in claim 24 wherein first distal section has a higher stiffness than both the proximal section and the second distal section.

27. The guide wire as claimed in claim 24 wherein a distal portion of the second distal section has a j-hook shape.

28. The guide wire as claimed in claim 24 wherein a distal portion of the second distal section has a hockey-stick shape.

29. A guide wire comprising a proximal section, a first distal section, a second distal section, and distal third distal section terminating in a tip, the first distal section being distal to the proximal section, the second distal section being distal to the first distal section, the third distal section being distal to the second distal section, said third distal section having a larger cross sectional area than the second distal section, said first distal section having a larger cross sectional area than the proximal section and the second distal section.

30. The guide wire as claimed in claim 29 wherein the third distal section has a higher stiffness than the second distal section and wherein the first distal section has a higher stiffness than the proximal section and the second distal section.

31. The guide wire as claimed in claim 29 wherein a distal portion of the third distal section has a j-hook shape.

32. The guide wire as claimed in claim 29 wherein a distal portion of the third distal section has a hockey-stick shape.
